(19) United States
(12) Patent Application Publication
(10) Pub. No.: US 2019/0392927 A1
(43) Pub. Date: Dec. 26, 2019

(54) PROCESS FOR PERSONALIZING BEHAVIOR TREATMENT PLANS TO CURE CHRONIC DISEASE

(71) Applicant: Rosemary Ku, San Francisco, CA (US)

(72) Inventor: Rosemary Ku, San Francisco, CA (US)

(21) Appl. No.: 16/448,479

(22) Filed: Jun. 21, 2019

Related U.S. Application Data
(60) Provisional application No. 62/689,183, filed on Jun. 24, 2018.

(51) Int. Cl. G16H 10/60; A61B 5/00; G16H 50/20; G16H 50/30; G16H 50/80; G16H 80/00; G16H 10/20; A61B 5/02; A61B 5/145

(52) U.S. Cl. CPC G16H 10/60; A61B 5/0002; G16H 50/20; G16H 50/30; G16H 50/80; G16H 20/10; G16H 10/20; A61B 5/02; A61B 5/14532; A61B 5/4076; G16H 80/00

(57) ABSTRACT

The invention is a process for integrating the science of chronic disease reversal with individual health and psychosocial data to generate personalized treatment plans to reverse a user's chronic condition(s). The user is an individual who has been diagnosed with a chronic disease and the treatment plans are delivered via web and mobile user interfaces. The treatment plans provide daily guidance on strategic behaviors, supplementary information on how those behaviors impact disease, and how progress is measured. In order to optimize clinical effectiveness of the treatment plans, the recommendations are not static but rather dynamic and iterate based on new user data and feedback loops from compliance and disease monitoring. Data for these feedback loops are collected objectively through devices or by self-report through the user interface.

### PRIORITY CLAIM

The present application claims priority to Provisional Application No. 62/689,183, filed Jun. 24, 2018, titled Process for Personalizing Behavior Treatment Plans to Cure Chronic Disease.

### FIELD OF THE INVENTION

The present invention relates generally to generating personalized treatments to reverse chronic conditions, and more particularly, to a system and method for implementing data-driven development plans to cure chronic disease and maintain health.

### BACKGROUND OF THE INVENTION

Currently, the healthcare industry has challenges in managing chronic diseases, including diabetes and hypertension. Specifically, lack of real-time patient monitoring and lack of patient education contribute to the problems involved. Further, patients who are not properly monitored can reach critical stages and become seriously ill. Increased medical and insurance costs are a direct result.

The majority of adults in the United States now have at least one chronic health condition and many have two or more. These diseases are not only costly but contribute to most preventable deaths. Chronic conditions are generally treated with medications even though the underlying causes are mainly attributed to lifestyle. Unfortunately, the majority of people with chronic conditions do not take their medications as prescribed. None of these medications are curative and chronic diseases typically progress to complications and organ damage. In addition, systems that currently exist are utilized by patients and clinicians to manage a population of patients, or are systems equally applicable to various types of chronic diseases.

Diseases, such as hypertension, lipid disorders, diabetes, mood and anxiety disorders, which are amongst the most prevalent chronic conditions are potentially reversible without medications or surgical procedures. Although the science of reversing disease conditions exists, it is not accessible to the general public. Moreover, translating that science into practical daily behaviors is a challenge for most people unless they have specialized physicians or personal health coaches. In addition, there is considerable variability in individual physiology and psychology, and generalized recommendations are at best incomplete and at worst counterproductive.

Since the concept of chronic disease reversal is not yet widely adopted by the medical community, existing solutions have focused on chronic disease management with the aims of controlling symptoms and preventing complications rather than disease reversal. These solutions include systems for care coordination with providers, delivery of patient education, remote monitoring of disease markers and symptoms, and data analytics platforms to assess population health. Solutions for lifestyle management of chronic conditions have typically focused on provider dictated diet and exercise plans or self-selected care plans based on generalized recommendations.

A need exists to resolve chronic conditions and reverse chronic disease through alternative intervention. The system and method will address specific chronic disease conditions, have learning capabilities to target various specific chronic conditions, and have a personalized health approach applicable to an individual patient. The system will desirably be a scalable personalized solution to reverse multiple chronic conditions.

The following will beneficially detail the possibilities of solving such needs and implementing the technological measures to achieve the same.

### SUMMARY OF THE INVENTION

The following embodiments disclosed include a system and process for integrating the science of chronic disease reversal with individual health and psychosocial data to generate personalized treatment plans to reverse a user's chronic conditions. The user is an individual who has been diagnosed with a chronic disease and the treatment plans are delivered via web and/or mobile user interfaces. The treatment plans provide daily guidance on strategic behaviors, supplementary information on how those behaviors impact disease, and how progress is measured. In order to optimize clinical effectiveness of the treatment plans, the recommendations are not static but rather dynamic and iterate based on new user data and feedback loops from compliance and disease monitoring. Data for these feedback loops are collected objectively when possible through devices or by self-reporting through the user interface.

One embodiment disclosed herein includes a process for personalizing behavior treatment plans, comprising one or more steps including: (a) entering information from a patient user into a database of patient user information including a medical condition; (b) providing a real-time observation period to collect data; (c) implementing one or more data-driven behavioral strategies as part of an initial treatment plan; (d) monitoring the patient user to create an improved condition to designate an active treatment; and (e) providing an iterative treatment plan to maintain or cure the medical condition; wherein the iterative treatment plan to maintain or cure the medical condition is determined by data analytics that support the active treatment that creates the improved condition.

The medical condition may include two or more medical conditions. In addition, two or more medical conditions comprise chronic conditions. The behavior strategies are personalized behavioral strategies determined by a patient user data analytics recorded in the iterative treatment plan such that the personalized behavioral strategies target reversing one or more chronic conditions.

In one aspect, the process further comprises a step of generating the data-driven behavioral strategies from a database of validated strategies. In another aspect, the process further comprises a step of iterating on the personalized behavioral strategies to reverse chronic disease conditions. The step of monitoring the patient user includes determining an active treatment plan as based on the improved condition. The iterative treatment plan is a treatment matrix of algorithmically determined patterns from the active treatment plan that improve the medical condition.

In one embodiment, the treatment matrix allocates one or more treatment protocols within one or more lifestyle categories of the patient user. In another embodiment the treatment matrix comprises at least a first treatment delivered within a first lifestyle category and sequentially through at least a second lifestyle category. The treatment matrix may comprise a plurality of treatment protocols delivered within at least a first lifestyle category. The iterative treatment plan comprises patient user data that is implemented into the active treatment plan and the iterative treatment plan selects an intended behavior, communicates the intended behavior to the patient user, and monitors markers of the medical condition. In one aspect, a plurality of medical conditions are targeted in a treatment matrix of the iterative treatment plan.

Embodiments of the medical monitoring system disclosed herein personalize behavior treatment plans, the system comprising: a database of patient user information including a plurality of identifiers and medical conditions; a real-time observation platform to collect data; a secondary database of one or more data-driven behavioral strategies as implemented in a treatment plan aligned to at least a first medical condition; an active treatment that creates an improved condition; and an iterative treatment plan that incorporates the active treatment in a treatment matrix protocol to maintain or cure the medical condition; wherein the iterative treatment plan to maintain or cure the medical condition is determined by data analytics that support the active treatment. The plurality of identifiers and medical conditions include one or more of psychosocial information, behavioral data, physiological data, demographic information, among others. The active treatment reverses a specified pathophysiology.

In one aspect, a medical monitoring device or application that monitors and communicates the medical matrix protocol to the patient user is incorporated to allow installation of coding and programming of algorithms and software to manage data and integrate with connected and internet/cloud-based databases. In another aspect, the medical monitoring device is integrated with a wearable of the patient user with additional capabilities including connection to hardware, software, and cloud-based services, wireless connectivity, or otherwise. The application may be a computer-based application, mobile-app or software install, for exemplary purposes and without limitation, and integrated with the analytical components of a software program within or among personal computing device(s). The iterative treatment plan stores personalized data of the patient user and alerts a medical provider as to the status of the patient user.

Various combinations of the application and monitoring process may be applicable within and external to medicine in applying personalized behavioral treatment plans to cure chronic disease. Such combinations may be varied and altered as would be known by one skilled in the art.

### BRIEF DESCRIPTION OF THE DRAWINGS

The accompanying drawings, which are incorporated in and constitute a part of this specification, illustrate various embodiments of the present invention, and, together with the description, serve to explain the principles of the invention. The various features are not necessarily drawn to scale. In fact, the dimensions may be arbitrarily increased or decreased for clarity of discussion. In the drawings:

FIG. 1 is an exemplary embodiment relating to user experience.

FIG. 2 is an embodiment in reference to a single disease model.

FIG. 3 is an embodiment referencing a multiple disease model.

FIG. 4 (A, B, C) shows graphical depictions of embodiments of the technology as applied to treatment plan matrices.

FIG. 5 illustrates an embodiment encompassing feedback loops as integrated with user data.

### DETAILED DESCRIPTION

Reference will now be made in detail to the present embodiments of the invention, examples of which are illustrated in the accompanying drawings. Wherever possible, the same reference numbers will be used throughout the drawings to refer to the same or like parts.

The software-based system disclosed herein generates personalized treatment plans to reverse chronic conditions, specifically chronic disease conditions. Instead of relying on the patient or provider to dictate a treatment plan, the software-based system uses the latest science on disease reversal and individual data, alone or in combination, to prioritize strategic behaviors to improve health.

Embodiments further describe a process for automatic iteration of the treatment plans based on user data, and any new patient/user data, while implementing compliance and disease response feedback loops. The system and process provide a solution that is scalable to generate and iterate on personalized treatment plans to reverse multiple chronic conditions. The treatment plan is a step-by-step set of behavioral strategies tailored to an individual's medical and psychosocial data. Each component of the treatment plan has at least about four (4) basic parts: (i) an interventional behavior, (ii) an explanation of why that behavior helps reverse a person's chronic conditions, (iii) how to get started, and (iv) how progress is assessed by both process and outcome measures, among others. In addition to personalization of the interventional behavior, the explanation (ii) and how to get started (iii) sections may also be personalized to factors such as, but not limited to, a person's educational level, motivation factors, level of desire for detail, and baseline lifestyle. How progress is measured is determined by the specified interventional behavior.

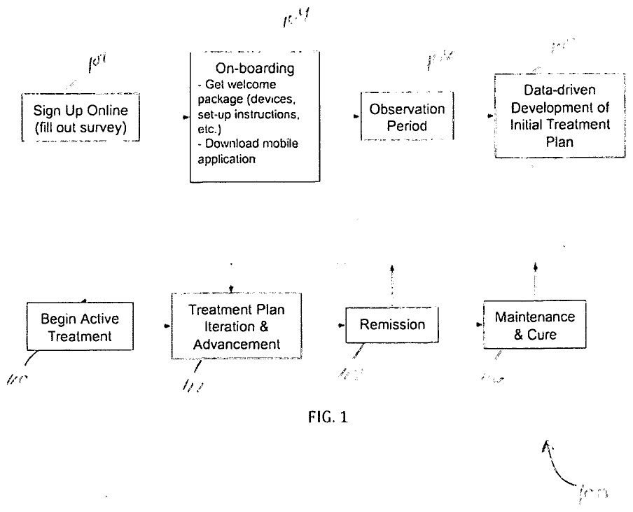

FIG. 1 depicts general user experience 100. Once an individual user signs up via an initial sign-up survey 102, he/she is on-boarded at a step 104. During such on-boarding 104, the user receives a welcome package including devices, set-up instructions, and information, as well as capability to download a mobile application or other integration data systems. Following on-boarding 104, the user goes through an observation period 106 to collect information about baseline behaviors and disease markers. Using data from the initial sign-up survey 102 and the observation period 106, the system generates data to determine an initial treatment plan 108. Data analytics and algorithms are implemented to facilitate a personalized initial treatment plan 108 and the user then commences the treatment phase 110. During this time, the treatment plan 108 adapts at step 112 to integrate a person's compliance and disease response. The integration at step 112 allows for treatment plan iteration and advancement using higher level algorithms and analytical support customized by the data pulled into the system and based on user behaviors. When the user reaches a remission phase 114, also referred to as a cure phase 114, the system reaches a step of maintaining the response and cure 116. Note that remission and cure are distinct stages. Remission is the normalization of disease markers off medication. Cure means that the user has been able to maintain remission for a specified amount of time. Clinical definitions for remission and length of time specified for cure are disease-dependent. Thus, the system can continue to monitor markers of disease and reinitiate treatment if a person's lifestyle changes or if the disease worsens.

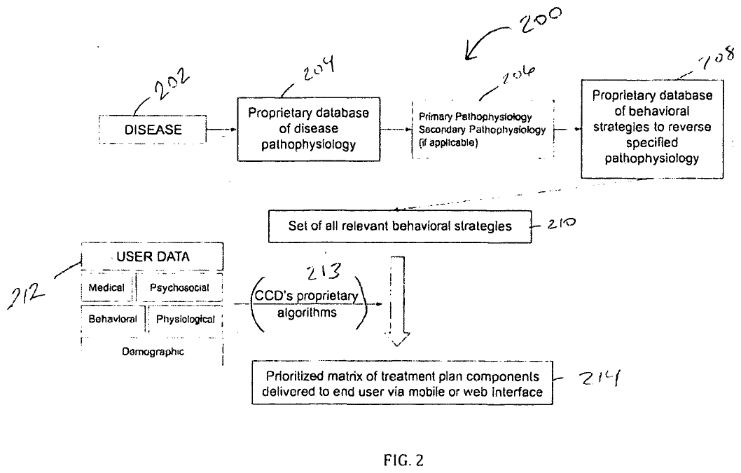

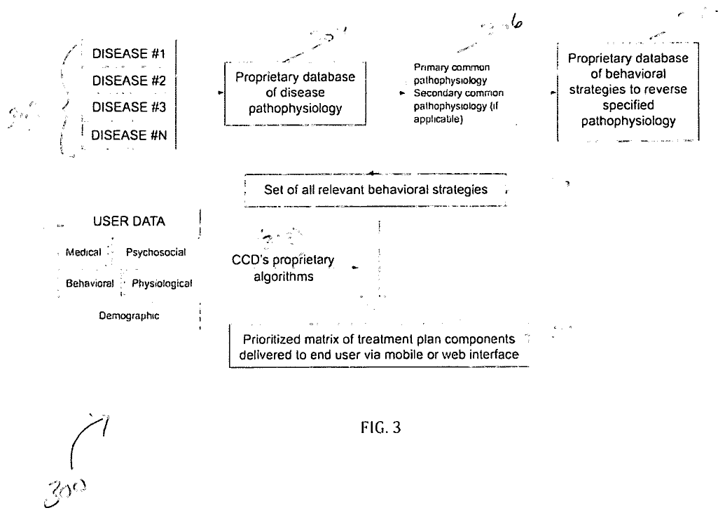

As shown in FIG. 2, the system 200 that develops an initial treatment plan is illustrated. The system for a single disease model 200 first recognizes at a step 202 a user's disease and identifies the underlying disease process 204, or pathophysiology, associated with the user's disease. In one aspect, the single disease model 200 is utilized. In another aspect, a set of diseases can be analyzed in a multiple disease model 300 (See FIG. 3) by querying the proprietary database of disease pathophysiology. For some conditions, more than one underlying disease process may be involved. If a user has more than one disease, a common pathophysiology for two or more of the person's conditions may be identified at a step 206. Many of the most common chronic conditions are physiologically related as in the case of diabetes and hypertension, which are both manifestations of the primary pathophysiology of insulin resistance. Then, based on the underlying disease processes identified at steps 204, 206, a query is placed into the proprietary database of evidence-based behavioral strategies known to reverse those processes at step 208. The output 210 is a set of behavioral strategies relevant to a person's disease (here, referencing the user). Identifying behavioral strategies 210 that target the underlying pathophysiology is a strategy to consolidate and eliminate redundancies in a treatment plan for multiple conditions that are interrelated. Development and maintenance of the databases may be done manually or automatically through software. Next, user data is utilized 212 using individual data across categories, including but not limited to medical, psychosocial, behavioral, physiological, and demographic data. Algorithms and data analytics 213 generate a prioritized matrix 214 of treatment plan components. Table 1 below provides examples of each of these categories and collection strategies but it is not exhaustive and more categories may be added over time.

In the multi-disease model 300 at FIG. 3, various diseases 302 are identified that pertain to a user and are recorded in a proprietary database 304 of disease pathophysiology. The primary common pathophysiology is recognized at step 306, along with any secondary common pathophysiology, if applicable. A query into a proprietary database 308 of behavioral strategies to reverse the specified pathophysiology will then identify relevant behavioral strategies 310 that target the underlying pathophysiology to determine a treatment plan 314. Here, the treatment plan 314 is a prioritized matrix of treatment plan components delivered to end user via mobile or web interface, the matrix of which has taken into account user data 311 data analytics 312 to implement the treatment plan 314. User data comprises medical, psychosocial, behavioral, physiological, and demographic data, among other variables as may be assessed and recorded from the user.

TABLE 1
User Data Categories

| | COLLECTION | |
|---|---|---|
| CATEGORY | STRATEGY | EXAMPLES |
| Medical | Self-report or | Diagnoses, medications, symptoms, |
| (physical or | EMR integration | diagnostic results (labs, imaging, |
| behavioral | | biometrics, etc.), risk factors |
| health) | | |
| Psychosocial | Self-report | Personality, social structure, |
| | | preferences, mindset |
| Behavioral | Self-report or | Sleep habits, dietary intake, |
| | monitoring | physical activity, self-care, |
| | devices | cognitive exercises |
| Physiological | Self-report or | Blood glucose, blood pressure, |
| (real-time) | monitoring | mood, pain |
| | devices | |
| Demographic | Self-report | Age, gender, geographical location, |
| | | occupation, education, income |

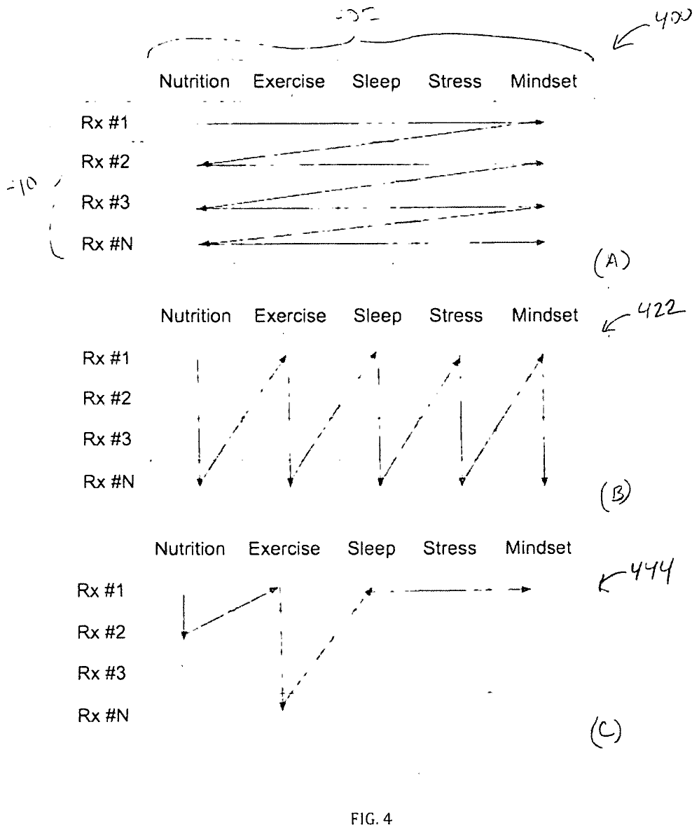

Examples of treatment matrices are shown in FIG. 4. The algorithms and methodology prioritizes both lifestyle categories and specific behavioral interventions (Rx) within each category, leading to a matrix 400 shown in FIG. 4. Major lifestyle areas 402 include, but are not limited to, nutrition, exercise, sleep, stress management, and mindset. Additional categories may be added over time, including for example medications. Prioritization of both the major lifestyle categories and components within each category (Rx) 410 is dynamic based on new data. The prioritization algorithms are based on several factors such as, but not limited to, lifestyle areas with greatest demand for improvement, expected physiological response, user compatibility or preference, or difficulty level of the intervention. The treatment plan may advance through the matrix based on compliance and/or disease response data, and the number of tasks (Rx) presented to each individual at a time may vary. This process can be altered manually or set to run automatically. Various paths of advancing through the matrix are depicted. FIG. 4A represents a treatment matrix that includes simultaneous application of treatment protocols 410, such that a first treatment protocol Rx #1 is followed throughout lifestyle categories 402, followed by a second treatment protocol Rx #2 throughout the lifestyle categories 402, and so on. FIG. 4B depicts a secondary matrix 422 such that each treatment protocol Rx #1, Rx #2, . . . Rx #N are followed within a lifestyle category 402, such as within a “nutrition” category, and then the same treatment protocols extended into a second lifestyle category such as “exercise”. At FIG. 4C, the matrix 444 creates a personalized delivery of treatment protocols 410 (e.g., Rx #1, Rx #2, . . . Rx #N) within each lifestyle category 402, as may be algorithmically determined by a set of user behaviors. The treatments are varied along with the lifestyle category of delivery as dependent on the behavior(s). New components may be added to the matrix at any time depending on improved scientific developments or modified user data, such components to include varied and improved treatment options, protocols, clinical studies. Lifestyle categories may also be modified at different stages of life, different stages of disease, treatment, specified behavior, cure, and response to treatment(s). One skilled in the art may vary such protocols and categories as desired in the system.

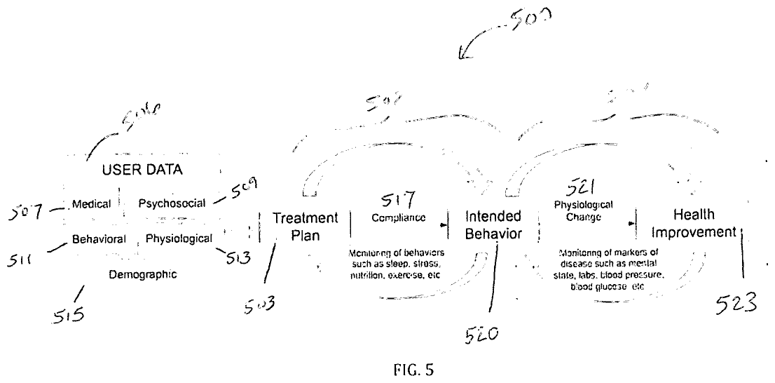

Embodiments disclosed here comprise at least two (2) major feedback loops, one feedback loop for compliance 502 and another feedback loop for physiological change 504, as demonstrated in FIG. 5. The feedback loops 502, 504 drive the iteration of and advancement through the treatment plan matrix 503. User data 506 for these feedback loops are collected objectively when possible through devices (e.g., data integration through Application Programming Interfaces (APIs)) or by self-report through the user interface (UI) when necessary or desired. Here, the user data comprises medical 507, psychosocial 509, behavioral 511, physiological 513 and demographic 515 data, among others as desired. The first feedback loop 502 is the intended behavior feedback loop, which measures compliance of the intended behavior 520 with the treatment plan. Examples of behavioral monitoring for compliance 517 include sleep and physical activity monitors and self-reported nutritional intake. This feedback loop assesses whether the algorithms and methodology correctly matched recommendations to a person's ability, motivation, preferences, and baseline lifestyle. Further, presenting compliance data to end-users provides positive reinforcement and builds self-efficacy.

The second feedback loop 504 is the health improvement feedback loop generated through disease monitoring. Examples of disease monitoring of the physiological change 521 include cellularly connected glucometers or blood pressure cuffs in the cases of diabetes and hypertension, respectively, and self-report of pain or mood. By frequently measuring disease response, the system captures real-time data on the effectiveness of the treatments plan(s) 503 as based on the health improvement 523 and can iterate on them, as desired, to achieve better results. Also, people might have different disease responses to the same or similar behavior. For instance, two people with diabetes might have very different blood glucose responses to the same food. This system allows for greater understanding of which interventions are most effective for specific user profiles. The feedback loops provide training sets for future machine learning to refine the treatment plan and algorithms associated therewith. Both data on compliance and disease response are made available to the end user through the web and mobile user interfaces.

If the user demonstrates an improvement in individual condition(s) regardless of compliance, the system runs a regression analysis on the user's behaviors to understand which behaviors are most correlated with health improvement. Results of this regression may be used to iterate the individual's treatment plan and/or system's algorithms and may be presented to the end user for positive reinforcement of healthy behaviors. If the user does not show an improvement but appears to be compliant with the treatment plan, the treatment plan may be advanced to a more aggressive intervention. If the user does not show an improvement and also is not compliant with the treatment plan, the system may go into troubleshooting mode. The user is then presented with a drop down menu of reasons why they did not complete the intended behavior (e.g. preference for different intervention, not enough time, lack of understanding, etc.) and the treatment plan adjusts based on reported reason. If the user does not engage with the troubleshooting mode, customer service representatives may reach out to better understand the situation.

Any modification of information or use of the above may include any number of variables be implemented and modified to achieve the same and does not depart from the spirit and scope of the disclosed invention.

What is claimed is:

1. A process for personalizing behavior treatment plans, comprising one or more steps including:
entering information from a patient user into a database of patient user information including a medical condition;
providing a real-time observation period to collect data;
implementing one or more data-driven behavioral strategies as part of an initial treatment plan;
monitoring the patient user to create an improved condition to designate an active treatment; and
providing an iterative treatment plan to maintain or cure the medical condition;
wherein the iterative treatment plan to maintain or cure the medical condition is determined by data analytics that support the active treatment that creates the improved condition.

2. The process of claim 1, wherein the medical condition includes two or more medical conditions.

3. The process of claim 2, wherein the two or more medical conditions comprise chronic conditions.

4. The process of claim 1, wherein the behavior strategies are personalized behavioral strategies determined by a patient user data analytics recorded in the iterative treatment plan such that the personalized behavioral strategies target reversing one or more chronic conditions.

5. The process of claim 4, further comprising a step of generating the data-driven behavioral strategies from a database of validated strategies.

6. The process of claim 5, further comprising a step of iterating on the personalized behavioral strategies to reverse chronic disease conditions.

7. The process of claim 1, wherein the step of monitoring the patient user determines an active treatment plan as based on the improved condition.

8. The process of claim 7, wherein the iterative treatment plan is a treatment matrix of algorithmically determined patterns from the active treatment plan that improve the medical condition.

9. The process of claim 7, wherein the treatment matrix allocates one or more treatment protocols within one or more lifestyle categories of the patient user.

10. The process of claim 9, wherein the treatment matrix comprises at least a first treatment delivered within a first lifestyle category and sequentially through at least a second lifestyle category.

11. The process of claim 9, wherein the treatment matrix comprises a plurality of treatment protocols delivered within at least a first lifestyle category.

12. The process of claim 1, wherein the iterative treatment plan comprises patient user data that is implemented into the active treatment plan and the iterative treatment plan selects an intended behavior, communicates the intended behavior to the patient user, and monitors markers of the medical condition.

13. The process of claim 12, wherein a plurality of medical conditions are targeted in a treatment matrix of the iterative treatment plan.

14. A medical monitoring system to personalize behavior treatment plans, comprising:
a database of patient user information including a plurality of identifiers and medical conditions;
a real-time observation platform to collect data;
a secondary database of one or more data-driven behavioral strategies as implemented in a treatment plan aligned to at least a first medical condition;
an active treatment that creates an improved condition; and
an iterative treatment plan that incorporates the active treatment in a treatment matrix protocol to maintain or cure the medical condition;
wherein the iterative treatment plan to maintain or cure the medical condition is determined by data analytics that support the active treatment.

15. The system of claim 14, wherein the plurality of identifiers and medical conditions include one or more of psychosocial information, behavioral data, physiological data, demographic information, among others.

16. The system of claim 15, wherein the active treatment reverses a specified pathophysiology.

17. The system of claim 14, further comprising a medical monitoring device or application that monitors and communicates the medical matrix protocol to the patient user.

18. The system of claim 17, wherein the medical monitoring device is integrated with a wearable of the patient user.

19. The system of claim 17, wherein the application is integrated with the analytical components of a software program within a personal computing device.

20. The system of claim 14, wherein the iterative treatment plan stores personalized data of the patient user and alerts a medical provider as to the status of the patient user.
